SEQUENCE LISTING (68 sequences)

SEQ ID NO: 1 - DNA, 345 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 2 - protein, 115 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 3 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 4 - protein, 107 aa, Artificial Sequence
  Synthesized
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 5 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcctcagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 6 - protein, 107 aa, Artificial Sequence
  Synthesized
DIQMTQSPSSLSTSVGDRVTLTCKASQDVGTAVDWYQQKPGLSPKLLIYWASTRHTGIPSRFSGSGSGTDFTLTISSLQPEDFADYYCQQYNSYPLTFGPGTKVDIK

SEQ ID NO: 7 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccacagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 8 - protein, 107 aa, Artificial Sequence
  Synthesized
DIQMTQSPSSLSTSVGDRVTLTCKASQDVGTAVDWYQQKPGHSPKLLIYWASTRHTGIPSRFSGSGSGTDFTLTISSLQPEDFADYYCQQYNSYPLTFGPGTKVDIK

SEQ ID NO: 9 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcaccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 10 - protein, 107 aa, Artificial Sequence
  Synthesized
DIQMTQSPSSLSTSVGDRVTLTCKASQDVGTAVDWYQQKPGTSPKLLIYWASTRHTGIPSRFSGSGSGTDFTLTISSLQPEDFADYYCQQYNSYPLTFGPGTKVDIK

SEQ ID NO: 11 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggctccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 12 - protein, 107 aa, Artificial Sequence
  Synthesized
DIQMTQSPSSLSTSVGDRVTLTCKASQDVGTAVDWYQQKPGSSPKLLIYWASTRHTGIPSRFSGSGSGTDFTLTISSLQPEDFADYYCQQYNSYPLTFGPGTKVDIK

SEQ ID NO: 13 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 14 - protein, 107 aa, Artificial Sequence
  Synthesized
DIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIK

SEQ ID NO: 15 - DNA, 711 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcggtggcggtggctcgggcggtggtgggtcgggtggcggcggatctgacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 16 - protein, 237 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGGGGSGGGGSGGGGSDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 17 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 18 - protein, 237 aa, Artificial Sequence
  Synthesized
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 19 - DNA, 711 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcggtggcggtggctcgggcggtggtgggtcgggtggcggcggatctgacatccagatgacccagagccccagcagcctgagcaccagcgtgggcgaccgcgtgaccctgacctgcaaggccagccaggacgtgggcaccgccgtggactggtaccagcagaagcccggcctcagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 20 - protein, 237 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGGGGSGGGGSGGGGSDIQMTQSPSSLSTSVGDRVTLTCKASQDVGTAVDWYQQKPGLSPKLLIYWASTRHTGIPSRFSGSGSGTDFTLTISSLQPEDFADYYCQQYNSYPLTFGPGTKVDIK

SEQ ID NO: 21 - DNA, 711 bp, Artificial Sequence; identical to SEQ ID NO: 19
  Synthesized

SEQ ID NO: 22 - protein, 237 aa, Artificial Sequence; identical to SEQ ID NO: 20
  Synthesized

SEQ ID NO: 23 - DNA, 711 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcggtggcggtggctcgggcggtggtgggtcgggtggcggcggatctgacatccagatgacccagagccccagcagcctgagcaccagcgtgggcgaccgcgtgaccctgacctgcaaggccagccaggacgtgggcaccgccgtggactggtaccagcagaagcccggcaccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 24 - protein, 237 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGGGGSGGGGSGGGGSDIQMTQSPSSLSTSVGDRVTLTCKASQDVGTAVDWYQQKPGTSPKLLIYWASTRHTGIPSRFSGSGSGTDFTLTISSLQPEDFADYYCQQYNSYPLTFGPGTKVDIK

SEQ ID NO: 25 - DNA, 711 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcggtggcggtggctcgggcggtggtgggtcgggtggcggcggatctgacatccagatgacccagagccccagcagcctgagcaccagcgtgggcgaccgcgtgaccctgacctgcaaggccagccaggacgtgggcaccgccgtggactggtaccagcagaagcccggctccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 26 - protein, 237 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGGGGSGGGGSGGGGSDIQMTQSPSSLSTSVGDRVTLTCKASQDVGTAVDWYQQKPGSSPKLLIYWASTRHTGIPSRFSGSGSGTDFTLTISSLQPEDFADYYCQQYNSYPLTFGPGTKVDIK

SEQ ID NO: 27 - DNA, 711 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcggtggcggtggctcgggcggtggtgggtcgggtggcggcggatctgacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 28 - protein, 237 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGGGGSGGGGSGGGGSDIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIK

SEQ ID NO: 29 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 30 - protein, 237 aa, Artificial Sequence
  Synthesized
DIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 31 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggctccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 32 - protein, 237 aa, Artificial Sequence
  Synthesized
DIQMTQSPSSLSTSVGDRVTLTCKASQDVGTAVDWYQQKPGSSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 33 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcaccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 34 - protein, 237 aa, Artificial Sequence
  Synthesized
DIQMTQSPSSLSTSVGDRVTLTCKASQDVGTAVDWYQQKPGTSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 35 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccacagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 36 - protein, 237 aa, Artificial Sequence
  Synthesized
DIQMTQSPSSLSTSVGDRVTLTCKASQDVGTAVDWYQQKPGHSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 37 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcctcagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 38 - protein, 237 aa, Artificial Sequence
  Synthesized
DIQMTQSPSSLSTSVGDRVTLTCKASQDVGTAVDWYQQKPGLSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 39 - DNA, 375 bp, Artificial Sequence
  Synthesized
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctct

SEQ ID NO: 40 - protein, 125 aa, Artificial Sequence
  Synthesized
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSS

SEQ ID NO: 41 - DNA, 330 bp, Artificial Sequence
  Synthesized
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaa

SEQ ID NO: 42 - protein, 110 aa, Artificial Sequence
  Synthesized
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLK

SEQ ID NO: 43 - DNA, 2088 bp, Artificial Sequence
  Synthesized
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 44 - protein, 696 aa, Artificial Sequence
  Synthesized
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSDIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 45 - DNA, 375 bp, Artificial Sequence; identical to SEQ ID NO: 39
  Synthesized

SEQ ID NO: 46 - protein, 125 aa, Artificial Sequence; identical to SEQ ID NO: 40
  Synthesized

SEQ ID NO: 47 - DNA, 1329 bp, Artificial Sequence
  Synthesized
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 443 aa, Artificial Sequence
  Synthesized
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - DNA, 1380 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 50 - protein, 460 aa, Artificial Sequence
  Synthesized
DIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 321 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcaagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaag

SEQ ID NO: 52 - protein, 107 aa, Artificial Sequence
  Synthesized
DIQMTQSPSSLSTSVGDRVTLTCKASQDVGTAVDWYQQKPGKSPKLLIYWASTRHTGIPSRFSGSGSGTDFTLTISSLQPEDFADYYCQQYNSYPLTFGPGTKVDIK

SEQ ID NO: 53 - DNA, 711 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggcaagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagggtggcggtggctcgggcggtggtgggtcgggtggcggcggatctgaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagc

SEQ ID NO: 54 - protein, 237 aa, Artificial Sequence
  Synthesized
DIQMTQSPSSLSTSVGDRVTLTCKASQDVGTAVDWYQQKPGKSPKLLIYWASTRHTGIPSRFSGSGSGTDFTLTISSLQPEDFADYYCQQYNSYPLTFGPGTKVDIKGGGGSGGGGSGGGGSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 55 - DNA, 711 bp, Artificial Sequence; identical to SEQ ID NO: 53
  Synthesized

SEQ ID NO: 56 - protein, 237 aa, Artificial Sequence; identical to SEQ ID NO: 54
  Synthesized

SEQ ID NO: 57 - DNA, 1725 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcgctagtacgaaaggtccggaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 58 - protein, 575 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 1686 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagactgttgctgcaccgtcagtgttcatcttccctccacaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - protein, 562 aa, Artificial Sequence
  Synthesized
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKTVAAPSVFIFPPQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - DNA, 1719 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccccagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagactgttgctgcaccgtcagtgttcatcttccctccagaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 62 - protein, 573 aa, Artificial Sequence
  Synthesized
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKTVAAPSVFIFPPEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1692 bp, Artificial Sequence
  Synthesized
gaggtgcagctggtgcagagcggccccgaggtgaagaagcccggcgccaccgtgaagatcagctgcaagaccagcggctacaccttcaccgagtacaccatccactgggtgaagcaggcccccggcaagggcctggagtggatcggcaacatcaaccccaacaacggcggcaccacctacaaccagaagttcgaggacaaggccaccctgaccgtggacaagagcaccgacaccgcctacatggagctgagcagcctgcgcagcgaggacaccgccgtgtactactgcgccgccggctggaacttcgactactggggccagggcaccctacttacagtaagtagcgctagtacgaaaggtccgcaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 64 - protein, 564 aa, Artificial Sequence
  Synthesized
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - DNA, 1686 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagactgttgctgcaccgtcagtgttcatcttccctccacaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 66 - protein, 562 aa, Artificial Sequence
  Synthesized
DIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIKTVAAPSVFIFPPQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - DNA, 1719 bp, Artificial Sequence
  Synthesized
gacatccagatgacccagagccccagcagcctgagcaccagcgtgggcgaccgcgtgaccctgacctgcaaggccagccaggacgtgggcaccgccgtggactggtaccagcagaagcccggccagagccccaagctgctgatctactgggccagcacccgccacaccggcatccccagccgcttcagcggcagcggcagcggcaccgacttcaccctgaccatcagcagcctgcagcccgaggacttcgccgactactactgccagcagtacaacagctaccccctgaccttcggccccggcaccaaggtggacatcaagactgttgctgcaccgtcagtgttcatcttccctccagaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - protein, 573 aa, Artificial Sequence
  Synthesized
DIQMTQSPSSLSTSVGDRVTLTCKASQDVGTAVDWYQQKPGQSPKLLIYWASTRHTGIPSRFSGSGSGTDFTLTISSLQPEDFADYYCQQYNSYPLTFGPGTKVDIKTVAAPSVFIFPPEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0411530 A1
(43) Pub. Date: Dec. 29, 2022

(54) PSMA Antibody and Use Thereof

(71) Applicant: NANTONG YICHEN BIOPHARMA. CO. LTD., Nantong (CN)

(72) Inventors: Feng WANG, Shanghai (CN); Huayang ZHENG, Shanghai (CN); Yuhan ZHANG, Shanghai (CN)

(21) Appl. No.: 17/778,422

(22) PCT Filed: Nov. 20, 2020
(86) PCT No.: PCT/CN2020/130511

§ 371 Date: May 20, 2022

(30) Foreign Application Priority Data
Nov. 22, 2019 (CN) 201911155025.8

(51) Int. Cl. C07K 16/30; C07K 16/28

(52) U.S. Cl. CPC C07K 16/30; C07K 16/2809; C07K 2317/56; C07K 2317/31

(57) ABSTRACT

The present disclosure relates to an antibody fragment specifically binding to PSMA, particularly to a single-chain Fv (scfv), a bispecific antibody containing the scFv, and a chimeric antigen receptor (CAR) and the preparation and the use thereof.

### CROSS-REFERENCE TO RELATED APPLICATIONS

The present application is a National Stage of International Patent Application No: PCT/CN2020/130511 filed on Nov. 20, 2020, which application claims the benefit of priority to the Chinese patent application No. 201911155025.8 filed on Nov. 22, 2019, which is herein incorporated by reference in their entirety.

### SEQUENCE LISTING

The present application contains a Sequence Listing which has been submitted electronically in ASCII format and is hereby incorporated by reference in its entirety. Said ASCII copy is named PN160534_Sequence_Listing.txt and is 124 kilobytes in size, and the sequences are identical to the sequence listing corresponding international application No. PCT/CN2020/130511 filed on Nov. 20, 2020, except that the source of all the artificial sequences has been amended to “Synthesized”, and the file reference number “PN160534YCYY” has been amended to the current reference number “PN148685BYZD”, no new matter is added.

### TECHNICAL FIELD

The present disclosure provides an antibody fragment specifically binding to PSMA, and particularly relates to a single-chain Fv (scfv) specifically binding to PSMA, a bispecific antibody containing the scFv, and a chimeric antigen receptor (CAR) and the preparation and the use thereof.

### BACKGROUND

Prostatic cancer is the second leading cause of death in men, ranking only second to lung cancer. However, the current methods, for example, surgery, radiotherapy, chemotherapy and androgen deprivation therapy, have limited effects on those advanced cancers. Up to now, radical surgery is still the major treatment for prostatic cancer. Therefore, emerging precision medicine, particularly the precision medicine based on tumor-targeted antibodies is expected to improve the outcome for prostate cancer sufferers.

Prostate specific membrane antigen (PSMA) is mainly expressed by prostatic epithelial cells. In prostatic cancer, especially in poorly differentiated, metastatic, and hormone-resistant cancers, the expression of PSMA is markedly increased (Gregorakis A K, et al., (1998) Seminars in Urologic Oncology 16: 2-12; Silver, D A (1997) Clinical Cancer Research 3: 85-515). Low-level expression of PSMA has been found in extra-prostate tissues, such as small intestine, salivary glands, duodenum mucosa, proximal renal tubules and brain (Silver, D A (1997) Clinical Cancer Research 3: 85-515). PSMA is also expressed in the endothelial cells of capillaries in the peripheral and intratumor regions of some malignant tumors (including renal cell carcinoma and colorectal carcinoma), but not in the blood vessels of normal tissues. Moreover, it is reported that PSMA is associated with tumor angiogenesis (Silver, D. A. (1997) Clinical Cancer Research 3: 81-85). Recently, it has been proved that PSMA is expressed in the endothelial cells of tumor-associated neovasculature system in colorectal cancer, breast cancer, bladder cancer, pancreatic cancer, kidney cancer, and melanoma (Chang, S. S. (2004) Curr Opin Investig Drugs 5: 611-5), laying the foundation for developing prostatic cancer-targeted precision medicine.

### SUMMARY

The present disclosure screens and obtains a series of high-affinity antibodies against PSMA by phage display technology. The inventor further finds that in these antibodies presented in the form of scFv, the amino acids at position 42 of the light chain (VL) has remarkable influence on the affinity; further, the protein yield of bispecific antibodies containing VL with a specific amino acids at position 42 has been significantly improved. The aPSMA scfv antibody fragment in the present disclosure may be used to construct bispecific antibodies, chimeric antigen receptors and the like, meeting the affinity requirements for aPSMA scfv in the field of tumor targeted therapy.

In one aspect, the present disclosure, provides an antibody fragment that specifically binds human PSMA, including:

A) a heavy chain variable region (VH) with the amino acid sequence as shown in SEQ ID NO: 2;

B) a light chain variable region (VL) with the amino acid sequence selected from the followings: SEQ ID NO: 6, SEQ ID NO: 8, SEQ ID NO: 10, SEQ ID NO: 12, SEQ ID NO: 14 or SEQ ID NO: 52.

In some embodiments, the antibody fragment that specifically binds PSMA is a single-chain Fv (scfv). In some embodiments, the PSMA-targeted scfv has a VH with the amino acid sequence shown in SEQ ID NO: 2 and a VL with the amino acid sequence shown in SEQ ID NO: 6. In some embodiments, the PSMA-targeted scfv has a VH with the amino acid sequence shown in SEQ ID NO: 2 and a VL with the amino acid sequence shown in SEQ ID NO: 8. In some embodiments, the PSMA-targeted scfv has a VH with the amino acid sequence shown in SEQ ID NO: 2 and a VL with the amino acid sequence shown in SEQ ID NO: 10. In some embodiments, the PSMA-targeted scfv has a VH with the amino acid sequence shown in SEQ ID NO: 2 and a VL with the amino acid sequence shown in SEQ ID NO: 12. In some embodiments, the PSMA-targeted scfv has a VH with the amino acid sequence shown in SEQ ID NO: 2 and a VL with the amino acid sequence shown in SEQ ID NO: 14. In some embodiments, the PMSA-targeted scfv has a VH with the amino acid sequence shown in SEQ ID NO: 2 and a VL with the amino acid sequence shown in SEQ ID NO: 52.

In some embodiments, the PSMA-targeted scfv has an amino acid sequence as shown in SEQ ID NO: 20. In some embodiments, the PSMA-targeted scfv has an amino acid sequence as shown in SEQ ID NO: 22. In some embodiments, the PSMA-targeted scfv has an amino acid sequence as shown in SEQ ID NO: 24. In some embodiments, the PSMA-targeted scfv has an amino acid sequence as shown in SEQ ID NO: 26. In some embodiments, the PSMA-targeted scfv has an amino acid sequence as shown in SEQ ID NO: 28. In some embodiments, the PSMA-targeted scfv has an amino acid sequence as shown in SEQ ID NO: 30. In some embodiments, the PSMA-targeted scfv has an amino acid sequence as shown in SEQ ID NO: 32. In some embodiments, the PSMA-targeted scfv has an amino acid sequence as shown in SEQ ID NO: 34. In some embodiments, the PSMA-targeted scfv has an amino acid sequence as shown in SEQ ID NO: 36. In some embodiments, the PSMA-targeted scfv has an amino acid sequence as shown in SEQ ID NO: 38. In some embodiments, the PSMA-targeted scfv has an amino acid sequence as shown in SEQ ID NO: 54. In some embodiments, the PSMA-targeted scfv has an amino acid sequence as shown in SEQ ID NO: 56.

In one aspect, the present disclosure provides a bispecific antibody, wherein the bispecific antibody including:

1) a first antigen binding domain specifically binding to PSMA, including:

A) a heavy chain variable region (VH) having an amino acid sequence as shown in SEQ ID NO: 2; and

B) a light chain variable region (VL) having an amino acid sequence selected from the following: SEQ ID NO: 6, SEQ ID NO: 8, SEQ ID NO: 10, SEQ ID NO: 12, SEQ ID NO: 14 or SEQ ID NO: 52, and

2) a second antigen binding domain.

In some embodiments, the first antigen binding domain of the bispecific antibody is a single-chain Fv (scfv) specifically binding to PSMA. In some embodiments, the first antigen binding domain of the bispecific antibody has an amino acid sequence as shown in SEQ ID NO: 20. In some embodiments, the first antigen binding domain of the bispecific antibody has an amino acid sequence as shown in SEQ ID NO: 22. In some embodiments, the first antigen binding domain of the bispecific antibody has an amino acid sequence as shown in SEQ ID NO: 24. In some embodiments, the first antigen binding domain of the bispecific antibody has an amino acid sequence as shown in SEQ ID NO: 26. In some embodiments, the first antigen binding domain of the bispecific antibody has an amino acid sequence as shown in SEQ ID NO: 28. In some embodiments, the first antigen binding domain of the bispecific antibody has an amino acid sequence as shown in SEQ ID NO: 30. In some embodiments, the first antigen binding domain of the bispecific antibody has an amino acid sequence as shown in SEQ ID NO: 32. In some embodiments, the first antigen binding domain of the bispecific antibody has an amino acid sequence as shown in SEQ ID NO: 34. In some embodiments, the first antigen binding domain of the bispecific antibody has an amino acid sequence as shown in SEQ ID NO: 36. In some embodiments, the first antigen binding domain of the bispecific antibody has an amino acid sequence as shown in SEQ ID NO: 38. In some embodiments, the first antigen binding domain of the bispecific antibody has an amino acid sequence as shown in SEQ ID NO: 54. In some embodiments, the first antigen binding domain of the bispecific antibody has an amino acid sequence as shown in SEQ ID NO: 56.

In some embodiments, the second antigen binding domain of the bispecific antibody binds to a specific receptor of T cells, wherein the specific receptor of T cells is preferably CD3. In some embodiments, the second antigen binding domain specifically binding to CD3 has a VH as shown in SEQ ID NO: 40 and a VL as shown in SEQ ID NO: 42.

In some embodiments, the bispecific antibody has sequences as shown in SEQ ID NO: 44 and SEQ ID NO: 46. In some embodiments, the bispecific antibody has sequences as shown in SEQ ID NO: 58 and SEQ ID NO: 60. In some embodiments, the bispecific antibody has sequences as shown in SEQ ID NO: 62 and SEQ ID NO: 64. In some embodiments, the bispecific antibody has sequences as shown in SEQ ID NO: 58 and SEQ ID NO: 66. In some embodiments, the bispecific antibody has sequences as shown in SEQ ID NO: 68 and SEQ ID NO: 64.

In one aspect, the present disclosure provides a chimeric antigen receptor (CAR), where the CAR comprises a scfv specifically binding to PSMA, a transmembrane domain and an intracellular domain.

In some embodiments, the PSMA-targeted scfv of the CAR has a VH as shown in SEQ ID NO: 2 and a VL as shown in SEQ ID NO: 6. In some embodiments, the PSMA-targeted scfv of the CAR has a VH as shown in SEQ ID NO: 2 and a VL as shown in SEQ ID NO: 8. In some embodiments, the PSMA-targeted scfv of the CAR has a VH as shown in SEQ ID NO: 2 and a VL as shown in SEQ ID NO: 10. In some embodiments, the PSMA-targeted scfv of the CAR has a VH as shown in SEQ ID NO: 2 and a VL as shown in SEQ ID NO: 12. In some embodiments, the PSMA-targeted scfv of the CAR has a VH as shown in SEQ ID NO: 2 and a VL as shown in SEQ ID NO: 14. In some embodiments, the PSMA-targeted scfv of the CAR has a VH as shown in SEQ ID NO: 2 and a VL as shown in SEQ ID NO: 52. In some embodiments, the PSMA-targeted scfv of the CAR has an amino acid sequence as shown in SEQ ID NO: 20. In some embodiments, the PSMA-targeted scfv of the CAR has an amino acid sequence as shown in SEQ ID NO: 22. In some embodiments, the PSMA-targeted scfv of the CAR has an amino acid sequence as shown in SEQ ID NO: 24. In some embodiments, the PSMA-targeted scfv of the CAR has an amino acid sequence as shown in SEQ ID NO: 26. In some embodiments, the PSMA-targeted scfv of the CAR has an amino acid sequence as shown in SEQ ID NO: 28. In some embodiments, the PSMA-targeted scfv of the CAR has an amino acid sequence as shown in SEQ ID NO: 32. In some embodiments, the PSMA-targeted scfv of the CAR has an amino acid sequence as shown in SEQ ID NO: 34. In some embodiments, the PSMA-targeted scfv of the CAR has an amino acid sequence as shown in SEQ ID NO: 36. In some embodiments, the PSMA-targeted scfv of the CAR has an amino acid sequence as shown in SEQ ID NO: 38. In some embodiments, the PSMA-targeted scfv of the CAR has an amino acid sequence as shown in SEQ ID NO: 54. In some embodiments, the PSMA-targeted scfv of the CAR has an amino acid sequence as shown in SEQ ID NO: 56. In some embodiments, the CAR has an amino acid sequence as shown in SEQ ID NO: 50.

In one aspect, the present disclosure provides a polynucleotide which encodes the antibody fragment, bispecific antibody or CAR that specifically binds to PSMA in any one of the preceding embodiments. In some embodiments, the polynucleotide comprises a nucleotide sequence selected from the following group: 5, 7, 9, 11, 13, 19, 21, 23, 25, 27, 29, 31, 33, 35, 37, 39, 41, 43, 44, 45, 47, 49, 51, 53, 55, 57, 59, 61, 63, 65, and 67.

In one aspect, the present disclosure provides a vector containing the preceding polynucleotide.

In one aspect, the present disclosure provides a cell comprising the preceding vector.

In one aspect, the present disclosure provides a composition containing the antibody fragment, the bispecific antibody, the CAR, the polynucleotide, the vector, or the cell of any one of the preceding items, and the composition further comprising a pharmaceutically acceptable carrier.

In one aspect, the present disclosure provides a method for treating a subject suffering from a disease associated with PSMA expression, including administering to the subject an effective amount of the composition.

In one respect, the present disclosure provides a method for diagnosing a disease associated with PSMA expression in a mammal, and the method comprising: using the antibody fragment to detect the binding to human PSMA in a tissue sample separated from the mammal, and thus the specific binding of the antibody fragment to the human PSMA in the tissue sample is indicative of a disease associated with PSMA expression in the mammal.

### BRIEF DESCRIPTION OF THE DRAWINGS

The drawings constituting the present application are used to provide a further understanding to the prevent disclosure. The exemplary embodiments and descriptions of the prevent disclosure are intended to explain the prevent disclosure, but not be construed as improper limitation to this disclosure. In the drawings:

FIG. 1 shows the ELISA result of aPSMA LH RM and RM2 monoclonal phage, wherein RM denotes the first round of panning, and RM2 denotes the second round of panning.

FIG. 2 shows the ELISA result of aPSMA HL monoclonal phage.

FIG. 3A shows the ELISA binding of an aPSMA scfv antibody with amino acid mutation at position 42 of VL to PSMA-his; and FIG. 3B shows the binding of a PSMA-targeted scfv-Fc antibody (on the 42nd amino acid mutation of a VL) to LnCap cells as detected by flow cytometry.

FIG. 4 shows the binding of bispecific antibodies to Jurkat T cells as detected by flow cytometry.

FIG. 5 shows the binding of bispecific antibodies to LnCap cells as detected by flow cytometry.

FIG. 6 shows the cytotoxicity of the bispecific antibody on LnCap cells by recruiting human T cells.

FIG. 7 shows the PK curve of the bispecific antibody in mice.

### DETAILED DESCRIPTION OF THE EMBODIMENTS

### Examples

It should be noted that, in the case of no conflict, the embodiments of the present application are merely examples for illustration, and are not intended to limit the present disclosure in any way.

### EXAMPLES

### Example 1 Phage Library Screening

aPSMA HL (SEQ ID NO: 37) and aPSMA LH (SEQ ID NO: 39) were synthesized, and respectively cloned into the phagemid vector, subjected to random mutation with GeneMorph II random mutagenesis (Agilent), and transformed into XL1-blue competent cells. When OD600=0.6-0.8, the transformed XL1-blue cells were infected with an helper phage M13K07. After 12 hours, the phage was collected and the phage titer (phage library capacity) was measured.

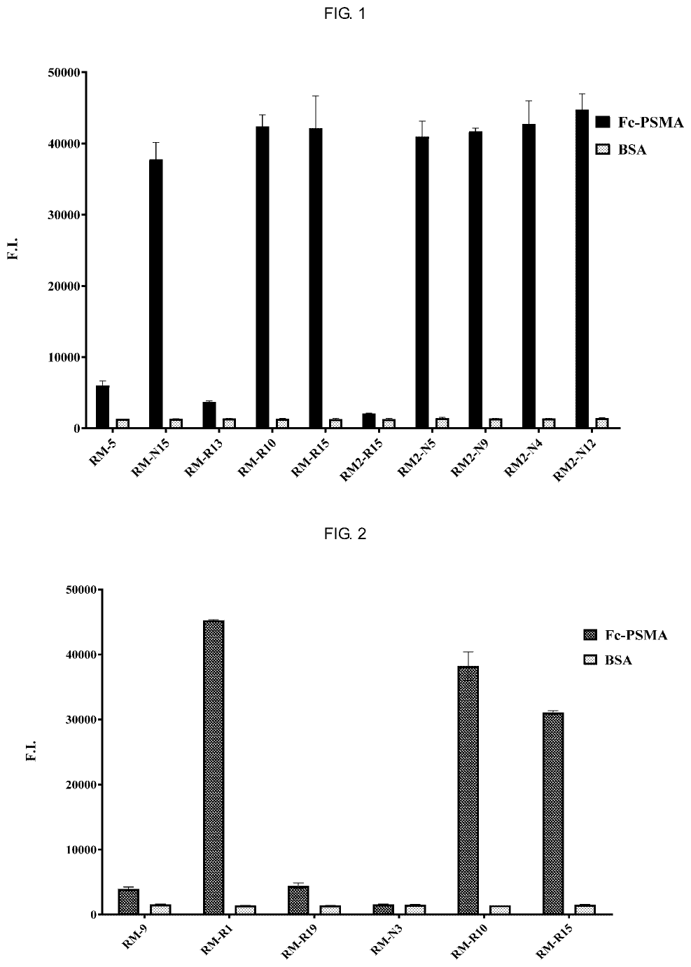

A microwell plate was coated with Fc-PSMA (300 ng/well) and blocked by BSA, the preceding two phage libraries were subjected to 2-3 rounds of panning and affinity detection, and single clones were picked randomly from the panned plates and sent for sequencing. Sequencing result analysis was performed. The single clones with sequence enrichment or mutations in the CDR regions were subjected to phage packaging and affinity detection. The detection result was shown in FIG. 1.

FIG. 1 shows the affinity detection result of the aPSMA LH RM and aPSMA LH RM2 mutant libraries. It can be seen from the figure that except for aPSMA LH RM-R13 and aPSMA LH RM2-R15, the affinity of other clones is much higher than that of the original sequence; FIG. 2 shows the affinity detection result of the aPSMA HL RM mutant library, and only aPSMA HL RM-R1/R10/R15 has a higher affinity.

After analyzing the above-mentioned sequences with higher affinity, it is found that in the aPSMA LH RM, aPSMA LH RM2 and aPSMA HL RM mutant libraries, the clones with higher affinity have a same amino acid mutation at the 42nd position of VL.

TABLE 1
Sequence Listing

| | | Nucleotide | Amino acid |
|---|---|---|---|
| | | sequence | sequence |
| | Description | SEQ ID NO: | SEQ ID NO: |
| aPSMA VH | VH | 1 | 2 |
| aPSMA VL | VL | 3 | 4 |
| aPSMA VL (L42) | VL | 5 | 6 |
| aPSMA VL (H42) | VL | 7 | 8 |
| aPSMA VL (T42) | VL | 9 | 10 |
| aPSMA VL (S42) | VL | 11 | 12 |
| aPSMA VL (Q42) | VL | 13 | 14 |
| aPSMA HL | scfv | 15 | 16 |
| aPSMA LH | scfv | 17 | 18 |
| aPSMA HL (L42) | scfv | 19 | 20 |
| aPSMA HL (H42) | scfv | 21 | 22 |
| aPSMA HL (T42) | scfv | 23 | 24 |
| aPSMA HL (S42) | scfv | 25 | 26 |
| aPSMA HL (Q42) | scfv | 27 | 28 |
| aPSMA LH (Q42) | Scfv | 29 | 30 |
| aPSMA LH (S42) | Scfv | 31 | 32 |
| aPSMA LH (T42) | Scfv | 33 | 34 |
| aPSMA LH (H42) | Scfv | 35 | 36 |
| aPSMA LH (L42) | Scfv | 37 | 38 |
| aCD3 VH | VH | 39 | 40 |
| aCD3 VL | VL | 41 | 42 |
| aCD3-aPSMA | Chain-1 | 43 | 44 |
| | Chain-2 | 45 | 46 |
| aCD3 | Chain-1 | 43 | 44 |
| | Chain-2 | 47 | 48 |
| aPSMA CAR | PSMA-targeted | 49 | 50 |
| | chimeric antigen | | |
| | receptor (CAR) | | |
| aPSMA VL (K42) | VL | 51 | 52 |
| aPSMA HL (K42) | scfv | 53 | 54 |
| aPSMA LH (K42) | scfv | 55 | 56 |
| aCD3-aPSMA.2 | Chain-1 | / | 57 |
| | Chain-2 | / | 58 |
| aCD3-aPSMA.3 | Chain-1 | / | 59 |
| | Chain-2 | / | 60 |
| aCD3-aPSMA.4 | Chain-1 | / | 57 |
| | Chain-2 | / | 61 |
| aCD3-aPSMA.5 | Chain-1 | / | 62 |
| | Chain-2 | / | 60 |

### Example 2 Detection of the Binding Affinity of PSMA-Targeted Antibody

2.1 Cloning and Expression of PSMA-Targeted Scfv-Fc Antibody

aPSMA HL (SEQ ID NO: 15), aPSMA LH (SEQ ID NO: 17), aPSMA HL (L42) (SEQ ID NO: 19), aPSMA HL (H42) (SEQ ID NO: 21), aPSMA HL (T42) (SEQ ID NO: 23), aPSMA HL (S42) (SEQ ID NO: 25), aPSMA HL (Q42) (SEQ ID NO: 27) and aPSMA LH (Q42) (SEQ ID NO: 29) were cloned into the N-terminal of pFuse-hIgG1-Fc2 by traditional enzyme digestion and ligation methods, and subjected to sequencing for verification.

The constructed eukaryotic expression vector was transiently transfected into FreeStyle HEK293 cells (ThermoFisher), respectively: 28 ml FreeStyle HEK293 (3×10⁷ cell/ml) were seeded into a 125 ml cell culture flask, plasmids were diluted with 1 ml Opti-MEM (Invitrogen) and then added to 1 ml Opti-MEM containing 60 μl 293 Fectin (Invitrogen, Inc). After incubating at room temperature for 30 min, the plasmid-293fectin mixture was added to the cell culture, and then incubated at 125 rpm, 37° C., 5% CO2. Cell culture supernatant was collected at 48 h and 96 h after transfection, respectively, and purified by Protein A Resin (Genscript) according to the manufacturer's instruction. The purified proteins were analyzed by SDS-PAGE.

2.2 Affinity Detection of PSMA-Targeted Scfv-Fc Antibody Against PSMA-his

PSMA-His(Acro) (100 ng/well) was coated in a 96-well plate and incubated at 4° C. overnight. After blocked by PBST (0.5% Tween-20 in PBS) containing 2% skim milk powder for 1 h at room temperature, gradiently diluted scfv-Fc antibodies were added and incubated for 2 h at room temperature. After washed by PBST containing 2% skim milk powder for 4-5 times, anti-human Fc-HRP secondary antibody was added for incubation for 1 h at room temperature. After washed by PBST containing 2% skim milk powder for 4-5 times, TMB reagent (BioLegend, Cat. 421101) was added for color development, and readings at 650 nm (not termination) or 450 nm (termination) were recorded. Data was analyzed by nonlinear regression with specific binding model by Prizm Graphpad software.

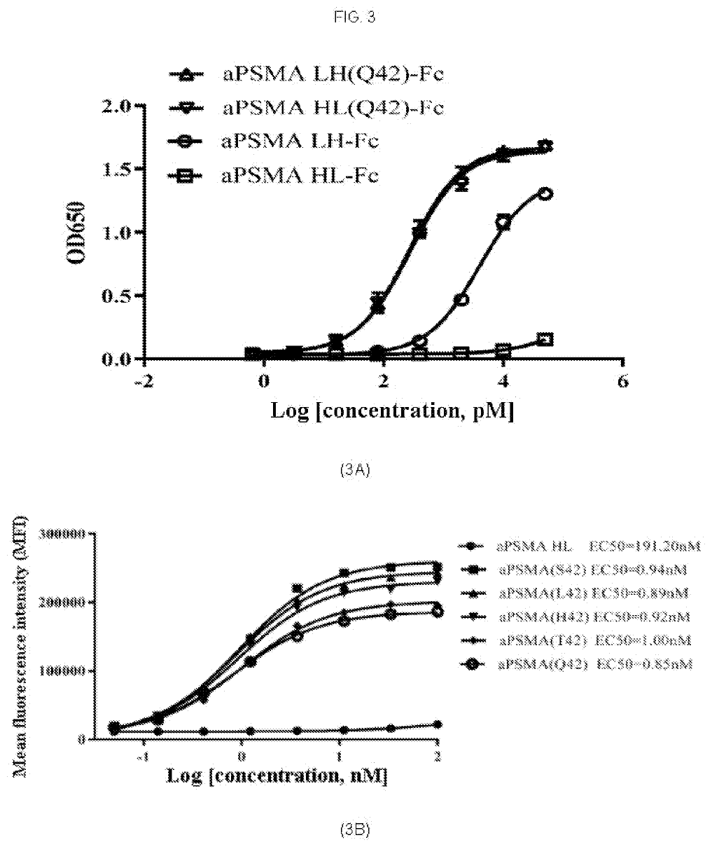

As shown in FIG. 3A, the aPSMA LH-Fc or aPSMA LH-Fc with wildtype VL has weak affinity agonist PSMA-his, while aPMSA HL (Q42)-Fc or aPSMA LH (Q42)-Fc with 42^{nd}-position amino acid mutation in VL has enhanced affinity to PSMA-his.

2.3 Binding Analysis of the PSMA-Targeted Scfv-Fc Antibody to LnCap Cells by Flow Cytometry

LnCap cells were cultured in RPMI 1640 medium containing 10% FBS in a 5% CO2 incubator. 2*10⁵ cells were washed for 3 times by pre-cooled PBS. After blocked by 2% FBS diluted by PBS, gradiently diluted antibodies (100 nM, gradiently diluted for 3 folds successively) were added and incubated for 1 hour at 4° C. Unbonded antibodies were washed away by 2% FBS. Mouse anti-human IgG Fc-APC (southern biotech) was added for 1 hour incubation at 4° C., and then subjected to flow cytometry after washed by 2% FBS. As shown in FIG. 3, the PSMA-targeted scfv-Fc with 42^{nd}-position amino acid mutation in VL has higher affinity against PSMA on the surface of LnCap cells, which is 190-220 times higher than that of PSMA-targeted scfv-Fc with wild type VL.

### Example 3 Bispecific Antibody Targeting PSMA and CD3

3.1 Construction and Expression of Bispecific Antibodies Targeting PSMA and CD3

Standard molecular biological methods were used to construct chain-1 and chain-2 containing different aCD3-aPMSA bispecific antibody as shown in Table 1 Sequence Listing, and subjected to sequencing for verification.

Eukaryotic expression vectors, containing the above-mentioned two chains, were verified by sequencing and transiently co-transfected into FreeStyle HEK293 cells (ThermoFisher) respectively: 28 ml FreeStyle HEK293 (3×10⁷ cell/ml) were seeded in a 125 ml cell culture flask, plasmids were diluted by 1 ml Opti-MEM (Invitrogen), and then added to 1 ml Opti-MEM containing 60 μl 293 Fectin (Invitrogen, Inc). After incubating at room temperature for 30 min, and the plasmid-293fectin mixture was added to the cell culture, and incubated at 125 rpm, 372, 5% CO2. Cell culture supernatant was collected at 48 hours and 96 hours after transfection, respectively, and purified by Protein A Resin (Genscript) according to the manufacturer's instruction, The purified proteins were analyzed by SDS-PAGE detection, and the yield was calculated. The results showed that the yield of aCD3-aPSMA bispecific antibody (15-20 mg/L) with 42^{nd}-position mutation in VL is significantly higher than that of aCD3-aPSMA bispecific antibody (2.5-5 mg/L) with a wild type VL.

3.2 In Vitro Activity Evaluation of Bispecific Antibody Targeting PSMA and CD3

3.2.1 Flow Cytometry Analysis on Binding to Jurkat (CD3+) Cells

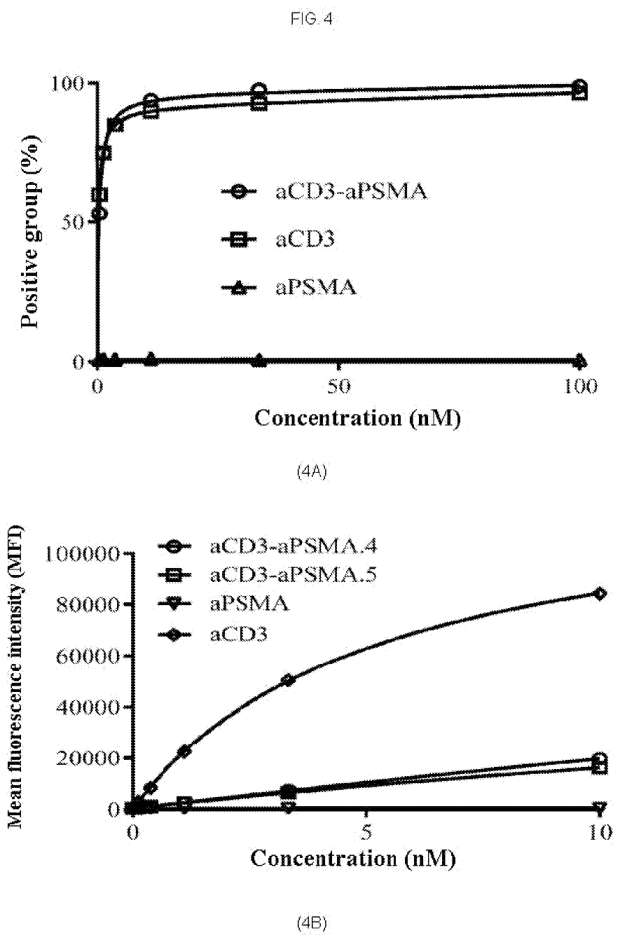

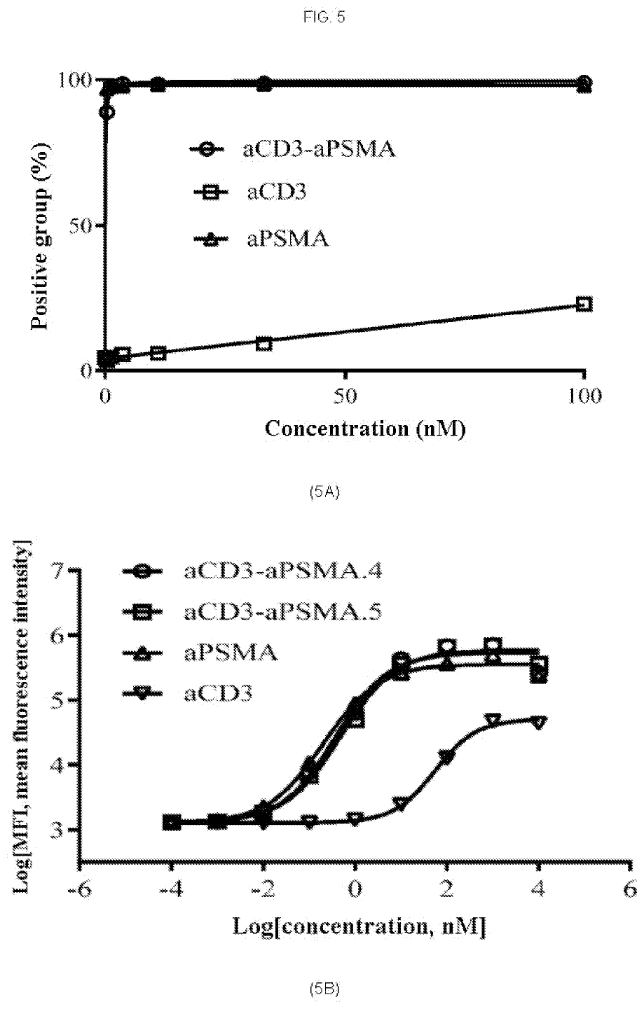

Jurkat cells were cultured in RPMI 1640 medium containing 10% FBS in a 5% CO2 incubator. 2×10⁵ cells were washed for 3 times with pre-cooled PBS. After blocked by PBS containing 2% FBS, bispecific antibody at concentrations of 100 nM, 33.3 nM, 11.1 nM, 3.7 nM, 1.2 nM or 0.4 nM was added and incubated for 1 hour at 4° C. Unbonded antibodies were washed by PBS containing 2% FBS, and mouse anti-human IgG Fc-APC (southern biotech) secondary antibody was added for 1 hour incubation at 4° C. After washing 3 times by PBS containing 2% FBS, flow cytometry was performed. As shown in FIG. 4, the bispecific antibody could effectively bind to Jurkat (CD3+) cells.

3.2.2 Flow Cytometry Analysis on Binding to LnCap (PSMA+) Cells

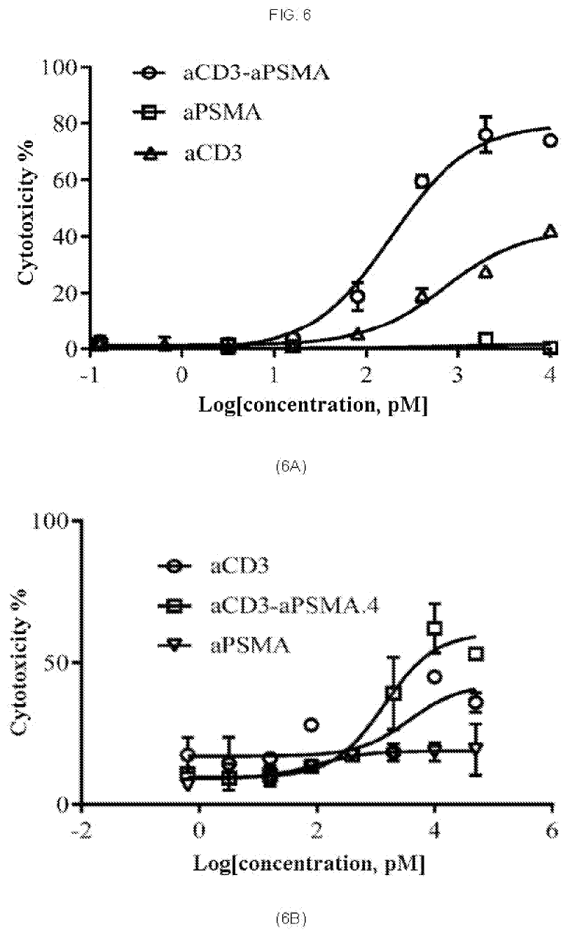

LnCap cells were cultured in RPMI 1640 medium containing 10% FBS in a 5% CO2 incubator. 2*10⁵ cells were washed for 3 times by pre-cooled PBS. After blocked by 2% FBS diluted by PBS, bispecific antibody at concentrations of 100 nM, 33.3 nM, 11.1 nM, 3.7 nM, 1.2 nM or 0.4 nM was added and incubated for 1 hour at 4° C. Unbonded antibodies were washed by 2% FBS, and mouse anti-human IgG Fc-APC (southern biotech) was added for incubation for 1 hour at 4° C. After washing by 2% FBS, flow cytometry was performed. As shown in FIG. 5, the bispecific antibody could effectively bind to LnCap cells.

TABLE 2
EC₅₀ of the bispecific antibody binding to Jurkat and LnCap cells

| EC₅₀ (nM) | aCD3-aPSMA | aCD3 | aPSMA |
|---|---|---|---|
| Jurkat | 0.51 | 0.51 | NA |
| LnCap | 0.05 | NA | 0.01 |

3.2.3 Detection of Human T Cell-Dependent Cytotoxicity

Peripheral blood was collected from healthy volunteer donor and peripheral blood mononuclear cells (PBMCs) were isolated by Ficoll-Hypaque (GE Healthcare) gradient centrifugation. The isolated PBMC was washed and incubated in RPMI medium containing 10% (vol/vol) FBS for 1 h to remove adherent cells. After activation by anti-CD3 and anti-CD28, cells were expanded in IL-2 for 10 days.

1×10⁴ LnCap cells (a RPMI medium containing 5% FBS) were mixed with activated PBMCs at a ratio of 1:5, and then incubated with bispecific antibody at different dilutions for 16 h at 37° C. The content of lactic dehydrogenase (LDH) in supernatant was detected by Cytotox-96 nonradioactive cytotoxicity assay kit (Promega). The value obtained from wells only containing target cells treated by the lysate was the Target Cell Maximum LDH Release Control; the value obtained from wells containing both effector cells and target cells treated by PBS (vehicle) was Spontaneous LDH Release Control. Color development was performed as the manufacturer's instruction, and absorbance at 490 nm was recorded in BMG LAB TECH CLARIOstar microplate reader (Bio-Gene Technology Ltd.). cytotoxicity was calculated as follows: % cytotoxicity=(absorbance experimental−absorbance spontaneous average)/(absorbance maximum killing average−absorbance spontaneous average).

As shown in FIG. 6, aCD3-aPSMA bispecific antibody could effectively recruit T cells to exert killing effects on target cells LnCap when compared with the control aPSMA.

TABLE 3
Killing of the bispecific antibody on the
target cell LnCap via calling on PBMC

| | aCD3-aPSMA | aCD3 | aPSMA |
|---|---|---|---|
| LDH Killing EC₅₀ (pM) | 184.4 | 712.3 | NA |
| LDH max killing (pM) | 79.79 | 42.76 | NA |

3.3 PK Study on the PSMA and CD3-Targeted Bispecific Antibody

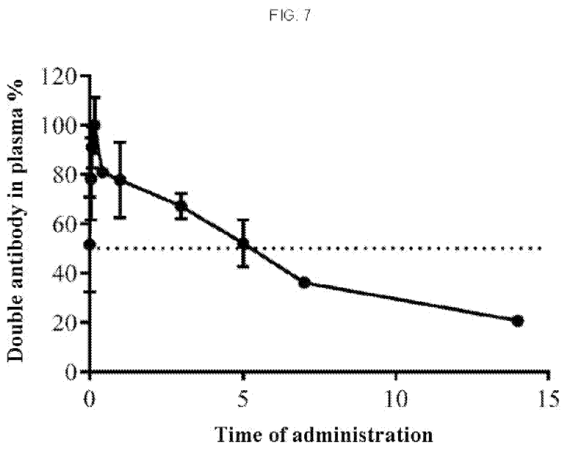

The bispecific antibody was intraperitoneally injected (I.P.) into C57 female mice (3 pieces/group, at a dose of 10 mg/kg). Whole blood was collected 30 min, 1 h, 2 h, 4 h, 10 h, 24 h, 3 d, 5 d, 7 d, and 14 d after injection. After centrifugation, plasma was collected and kept at −80° C. for further use. The bispecific antibody in the plasma was detected with reference to the Example 2.2. As shown in FIG. 7, the half-life of the bispecific antibody in mice is about 5 d.

1. An antibody fragment specifically binding to human PSMA, comprising:
A) a heavy chain variable region with an amino acid sequence as shown in SEQ ID NO: 2; and
B) a light chain variable region with an amino acid sequence selected from the followings: SEQ ID NO: 6, SEQ ID NO: 8, SEQ ID NO: 10, SEQ ID NO: 12, SEQ ID NO: 14 or SEQ ID NO: 52.

2. The antibody fragment according to claim 1, wherein the antibody fragment is a single-chain Fv.

3. The antibody fragment according to claim 2, wherein the single-chain Fv has an amino acid sequence as shown in SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 54 or SEQ ID NO: 56.

4. A bispecific antibody, the bispecific antibody comprising:
1) a first antigen binding domain specifically binding to PSMA, which is the antibody fragment specifically binding to human PSMA according to claim 1, comprising:
A) a heavy chain variable region with an amino acid sequence as shown in SEQ ID NO: 2; and
B) a light chain variable region with an amino acid sequence selected from the followings: SEQ ID NO: 6, SEQ ID NO: 8, SEQ ID NO: 10, SEQ ID NO: 12, SEQ ID NO: 14 or SEQ ID NO: 52, and
2) a second antigen binding domain.

5. The bispecific antibody according to claim 4, wherein the first antigen binding domain is a single-chain Fv, preferably, the single-chain Fv has an amino acid sequence as shown in SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 54 or SEQ ID NO: 56.

6. The bispecific antibody according to claim 4, wherein the second antigen binding domain binds to a specific acceptor molecule on T cells, and wherein the specific acceptor molecule on T cells is preferably CD3.

7. The bispecific antibody according to claim 6, wherein the second antigen binding domain specifically binding to CD3 has a heavy chain variable region as shown in SEQ ID NO: 40, and a light chain variable region as shown in SEQ ID NO: 42.

8. The bispecific antibody according to claim 4, comprising a group consisting of two polypeptide chains bindable to CD3 and PMSA: SEQ ID NO: 44 and SEQ ID NO: 46; SEQ ID NO: 58 and SEQ ID NO: 60; SEQ ID NO: 62 and SEQ ID NO: 64; SEQ ID NO: 58 and SEQ ID NO: 66; and SEQ ID NO: 68 and SEQ ID NO: 64.

9. A chimeric antigen receptor, comprising a single-chain Fv, specifically binding to PSMA, a membrane spanning domain and an intracellular domain, wherein the single-chain Fv, is the antibody fragment specifically binding to human PSMA according to claim 1, comprising:
A) a heavy chain variable region with an amino acid sequence as shown in SEQ ID NO: 2; and
B) a light chain variable region with an amino acid sequence selected from the following: SEQ ID NO: 6, SEQ ID NO: 8, SEQ ID NO: 10, SEQ ID NO: 12, SEQ ID NO: 14 or SEQ ID NO: 52.

10. The chimeric antigen receptor according to claim 9, having an amino acid sequence as shown in SEQ ID NO: 50.

11. A polynucleotide, encoding the antibody fragment according to claim 1.

12. The polynucleotide according to claim 11, having a nucleotide sequence as shown in SEQ ID NOS: 19, 21, 23, 25, 27, 29, 31, 33, 35, 37, 43, 45, 49, 51, 53, 55, 57, 59, 61, 63, 65 or 67.

13. A vector, comprising the polynucleotide according to claim 11.

14. A cell, comprising the polynucleotide according to claim 11.

15. The cell according to claim 14, wherein the cell is a T cell.

16. A composition, comprising the antibody fragment according to claim 1.

17. The composition of claim 16, further comprising a pharmaceutically acceptable carrier.

18. The composition of claim 16, used for chimeric antigen receptor T-cell immunotherapy.

19. A method for treating a subject suffering from a disease associated with PSMA expression, comprising administering to the subject an effective amount of the composition according to claim 16.

20. A method for diagnosing a disease associated with PSMA expression in a mammal, wherein the method comprises: using the antibody fragment according to claim 1 to detect the binding to human PSMA in a tissue sample separated from the mammal, and thus the specific binding of the antibody fragment to the human PSMA in the tissue sample is indicative of a disease associated with PSMA expression in the mammal.
